(19) United States
(12) Patent Application Publication
(10) Pub. No.: US 2014/0065235 A1
(43) Pub. Date: Mar. 6, 2014

(54) SEXUAL FUNCTION ENHANCEMENT COMPOSITION MADE FROM URECHIS UNICINCTUS EXTRACT CONTAINED AS EFFECTIVE COMPONENT MANUFACTURED BY WAY OF HIGH TEMPERATURE AND HIGH PRESSURE PROCESS AND ULTRASONIC WAVE PROCESS

(71) Applicant: PUKYONG NATIONAL UNIVERSITY INDUSTRY-UNIVERSITY CO, Busan (KR)

(72) Inventors: Se-Kwon KIM, Busan (KR); Bo-Mi Ryu, Busan (KR)

(73) Assignee: Pukyong National University Industry-University Cooperation Foundation, Busan (KR)

(21) Appl. No.: 13/665,586

(22) Filed: Oct. 31, 2012

(30) Foreign Application Priority Data
Sep. 3, 2012 (KR) 10-2012-0096912

(51) Int. Cl. A61K 35/62; A23L 1/29

(52) U.S. Cl. CPC A61K 35/62; A23L 1/296
USPC 424/520

(57) ABSTRACT

Disclosed are a pharmaceutical composition for a sexual function improvement, an erection dysfunction therapy agent containing the urechis unicinctus as an effective component, a health function food for a sexual function improvement and a method for preparing an urechis unicinctus extract with a sexual function improvement activation each of which contains an urechis unicinctus extract which is added as an effective component and yielded by way of a process at a temperature 100˜130° C. at a pressure of 1 Kgf/cm² for 20˜40 minutes and then with an ultrasonic wave at a frequency 35˜45 KHz for 50˜70 minutes.

### TECHNICAL FIELD

The present invention relates to a sexual function enhancement composition made from an urechis unicinctus extract contained as an effective component manufactured by way of a high temperature and high pressure process and an ultrasonic wave process.

### BACKGROUND ART

A sexual reaction undergoes by way of a penis erection, a half-ejaculation and an ejaculation. The above mentioned sexual function is a somehow complicated biological reaction which appears in combination with an endocrine system, a blood vessel system, a nervous system, a mental system, etc. and seems to be a health index for a normal adult. If one of an endocrine system, a blood vessel system, a nervous system, a mental system, etc. has a problem in functions, there appears a disorder in a sexual function.

As for the disorder in the sexual functions, it is reported that the people in their thirties account for 39%, the fifties 49%, the sixties 58%, the seventies 67%. The patients suffering from the disorder in the sexual functions continue to increase thanks to the increase of adult diseases coming along with the expansion of average life span and the change in food and life, the increase of the industrial disaster due to the fast industrialization, the physical fatigue due to the complicated modern life, drug, alcohol drinks, smoking, etc.

Therefore, the typical medicine treatments as well as the health functional continue to increase in an attempt to improve the disorder of the sexual functions.

As for Korea, lots of foods have been used as energetic foods for long time to improve the sexual functions; however such foods are not proved to have sexual function improvements. In addition, no accurate evaluations on the effects of therapy are disclosed.

Meanwhile, the sexual function improvement health foods in Korea have growth double every year along with the growth in economy and have been considered as a new industry which yields a lot of values. Even though it yields a lot of values in the industry, a plenty of problems occur in terms of a people's health and customer thanks to a system, a management and a distribution. The nation's competitiveness is dropping because of some limits in a new development, so a new medicine and a new health function food which have proved to have a science-based improvement in a sexual function are urgently needed.

The present invention has been made after it is proved that an urechis unicinctus extract manufactured by way of a high temperature and high pressure process and an ultrasonic wave process has an excellent improvement in a sexual function.

### DISCLOSURE OF INVENTION

Accordingly, it is an object of the present invention to provide a method for treating an erectile dysfunction in a mammal in need thereof which comprises administering to said mammal an effective amount of an urechis unicinctus extract and yielded by way of a process at a temperature 100˜130° C. at a pressure of 1 Kgf/cm² for 20˜40 minutes and then with an ultrasonic wave at a frequency 35˜45 KHz for 50˜70 minutes.

It is another object of the present invention to provide a pharmaceutical urechis unicinctus extract contained as an effective component manufactured by way of a high temperature and high pressure process and an ultrasonic wave process which is stable in a human body and provides an excellent improvement in a sexual function.

It is another object of the present invention to provide an erectile dysfunction therapy agent which contains an urechis unicinctus extract contained as an effective component.

It is another object of the present invention to provide a health food having an improvement in a sexual function which contains an urechis unicinctus extract contained as an effective component.

In addition, it is another object of the present invention to provide a method for preparing an urechis unicinctus extract having an improvement in a sexual function which comprises a step for cutting urechis unicinctus into 0.8 cm˜1.0 cm, a step for pressurizing the cut urechis unicinctus with 1 Kgf/cm² at a temperature of 100˜130° C. for 20˜40 minutes, and a step for processing an ultrasonic wave at a frequency of 35˜45 KHz for 50˜70 minutes.

It is another object of the present invention to provide a method for curing an erectile dysfunction which comprises a step of administrating an urechis unicinctus extract to an erectile dysfunction object which excludes a human being.

To achieve the above objects, there is provided a pharmaceutical composition for a sexual function improvement, comprising an urechis unicinctus extract contained as an effective component and yielded by way of a process at a temperature 100˜130° C. at a pressure of 1 Kgf/cm² for 20˜40 minutes and then with an ultrasonic wave at a frequency 35˜45 KHz for 50˜70 minutes.

The urechis unicinctus extract is contained in the composition at a concentration of 1˜100 ug/ml.

To achieve the above objects, there is provided an erection dysfunction therapy agent, comprising an urechis unicinctus extract contained as an effective component and yielded by way of a process at a temperature 100˜130° C. at a pressure of 1 Kgf/cm² for 20˜40 minutes and then with an ultrasonic wave at a frequency 35˜45 KHz for 50˜70 minutes.

The formulation of the erection dysfunction therapy agent is provided in a form of powders, tablets, capsules, injection liquid, smooth capsules, pills or granule.

The urechis unicinctus extract is contained at a concentration of 1˜100 ug/ml.

To achieve the above objects, there is provided a health food for a sexual function improvement, comprising an urechis unicinctus extract is contained as an effective component and yielded by way of a process at a temperature 100˜130° C. at a pressure of 1 Kgf/cm² for 20˜40 minutes and then with an ultrasonic wave at a frequency 35˜45 KHz for 50˜70 minutes.

The urechis unicinctus is contained at a concentration of 1˜100 ug/ml.

To achieve the above objects, there is provided a method for preparing an urechis unicinctus having a sexual function improvement activation, comprising a step for cutting urechis unicinctus into pieces each of 0.8 cm˜1.0 cm; a step for processing the cut urechis unicinctus at a temperature of 100˜130° C. at a pressure of 1 Kgf/cm² for 20˜40 minutes; and a step for performing an ultrasonic wave process at a frequency of 35˜45 KHz for 50˜70 minutes.

There is further provides a step for medicating an urechis unicinctus extract produced by the method of claim 8 to an erection dysfunction object except for a human being.

### Advantageous Effects

The present invention is directed to a method for preparing an urechis unicinctus extract a sexual function improvement pharmaceutical composition which contains as an effective component an urechis unicinctus extract processed at a pressure 1 Kgf/cm² and a temperature of 100˜130° C. for 20˜40 is minutes and to perform an ultrasonic wave process at a frequency of 35˜45 KHz for 50˜70 minutes, an erectile dysfunction therapy agent, a sexual function improvement health function food and a sexual function improvement and activation. The urechis unicinctus extract which is an effective component for the purpose of a sexual function improvement according to the present invention is extracted by way of a high temperature and high pressure and an ultrasonic wave process and has function of increasing the generation of nitrogen monoxide and GTP which are involved in promoting and enhancing the erection of a man's penis and of increasing the secretion of Testosterone which is a male hormones, so the present invention is useful for the sake of the manufacture of a sexual function improvement medicine and a functional food.

### BRIEF DESCRIPTION OF DRAWINGS

FIG. 1 is a graph showing a molecule weight distribution degree of an urechis unicinctus extract 1a obtained by processing an urechis unicinctus by way of a high temperature and high pressure process and an urechis unicinctus extract 1b obtained by way of a high temperature and high pressure and an ultrasonic wave process.

FIG. 2 is a graph showing a result of an analysis on a nitrogen monoxide generation with respect to each extract which is obtained by is processing an urechis unicinctus extract and other marine products by way of a high temperature and high pressure and an ultrasonic wave process.

FIG. 3 is a graph showing a result of an analysis on the production of a nitrogen monoxide which is an index of an erection after an urechis unicinctus extract obtained by way of a high temperature and high pressure and an ultrasonic wave process is treated on cells for 4 and 7 days.

FIG. 4 is a view showing a result of an analysis on a GTP generation in cells after an urechis unicinctus extract obtained by way of a high temperature and high pressure and an ultrasonic wave process performed on the cells of a smooth muscle is processed by the concentration.

FIG. 5 is a view showing a result of a measurement on a secretion amount of Testosterone which is a male hormone after an urechis unicinctus extract obtained by way of a high temperature and high pressure and an ultrasonic wave process on the cells of a smooth muscle is processed by the concentration.

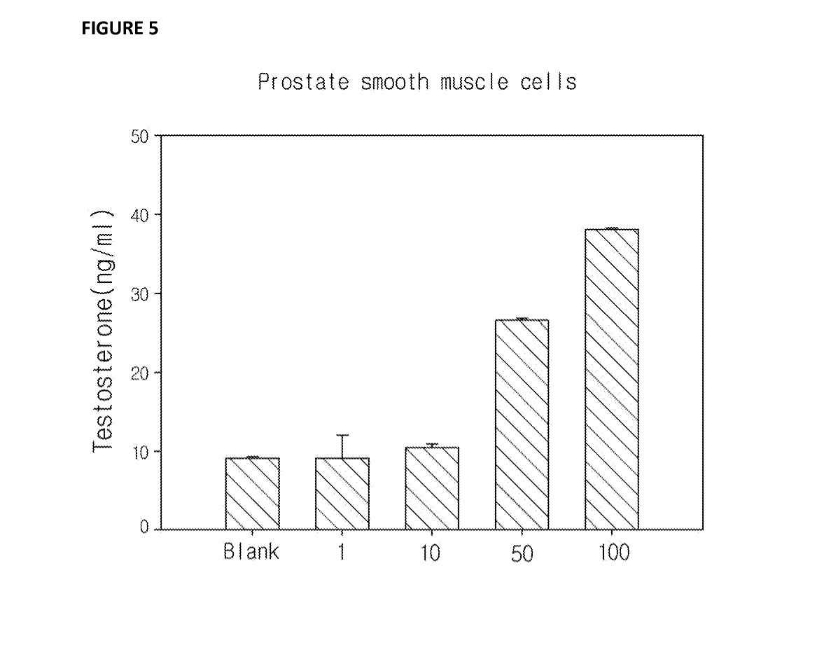

FIG. 6 is a view showing a result of the generation of a nitrogen monoxide based on a process of an urechis unicinctus extract while showing the urechis unicinctus samples: Raw which means that an urechis unicinctus sample is not processed, Autoclaved S which means that an urechis unicinctus sample is processed at only a high temperature and high pressure, and A+ sonicated S which means that an urechis unicinctus sample is processed at a high temperature and high pressure and with a ultrasonic wave.

### MODES FOR CARRYING OUT THE INVENTION

The present invention is characterized in that while we were studying a pharmaceutical substance having a good sexual function improvement effect and were trying to get it from the nature, it has been proved that an urechis unicinctus extract obtained by way of a high temperature and high pressure and an ultrasonic wave process has a good sexual function improvement effect.

The present invention has studies on urechis unicinctus which is one of a marine life so as to use it as a sexual function improvement agent. The urechis unicinctus extract lives on the shore of the Pacific Ocean in a U-shaped cave in the sand or mud. Since urechis unicinctus can prolong or contract the body, it is a little hard to find the urechis unicinctus featuring in a body length of 10˜15 cm and a thickness of 2˜4 cm with a mouth formed in a short, conical shape and pursing and extending by itself. 9˜13 rigid hairs grow near an anus, and the color of the body is a milk white color with a red color in part and it has a lot of protrusions on the body.

The urechis unicinctus contains lots of asparaginic acid which has proved to enhance alcohol metabolism, so it helps relieve hangover. It contains also a component which can dissolve thrombosis, so the urechis unicinctus is good for a hypertension patient.

As a method for obtaining an effective component having a good sexual function improvement effect from urechis unicinctus extract, there are a conventional method which is directed to obtaining an extract from the nature itself and a method which is directed to obtaining an urechis unicinctus extract which has been proved to have a sexual function improvement effect after it is processed at a high temperature and high pressure and with ultrasonic waves.

The present invention is directed to providing a method for preparing an urechis unicinctus extract which makes it possible to promote and enhance a sexual function effect, which method is characterized to comprise a step for cutting urechis unicinctus into pieces each of 0.8 cm˜1.0 cm; a step for processing the cut urechis unicinctus at a temperature of 100˜130° C. at a pressure of 1 Kgf/cm² for 20˜40 minutes; and a step for performing an ultrasonic wave process at a frequency of 35˜45 KHz for 50˜70 minutes.

The cut urechis unicinctus is processed at a high temperature and high pressure because of the sterilization of samples and because it is needed to obtain a stable state protein with the aid of a high temperature and high pressure because the protein component from the urechis unicinctus might be easily degraded. If the temperature and pressure exceeds the set ranges of temperature, time and pressure, the effective components having the sexual function enhancement activation contained in the urechis unicinctus might be destroyed. If it does not reach the set range of temperature, time and pressure, the effective components having the sexual function enhancement activation contained in the urechis unicinctus might not be extracted enough.

In addition, the ultrasonic wave process is performed after the high temperature and high pressure process is completed for the reasons why the protein contained in the urechis unicinctus can be decomposed to a small unit of low molecular peptides for the sake of easier degradation in the body. If the frequency and time are out of the set ranges of frequency and time, it seems to be out of the high temperature and high pressure, so some problems might occur.

So, it is preferred that the high temperature and high pressure and the ultrasonic wave processes are performed in the above mentioned ranges.

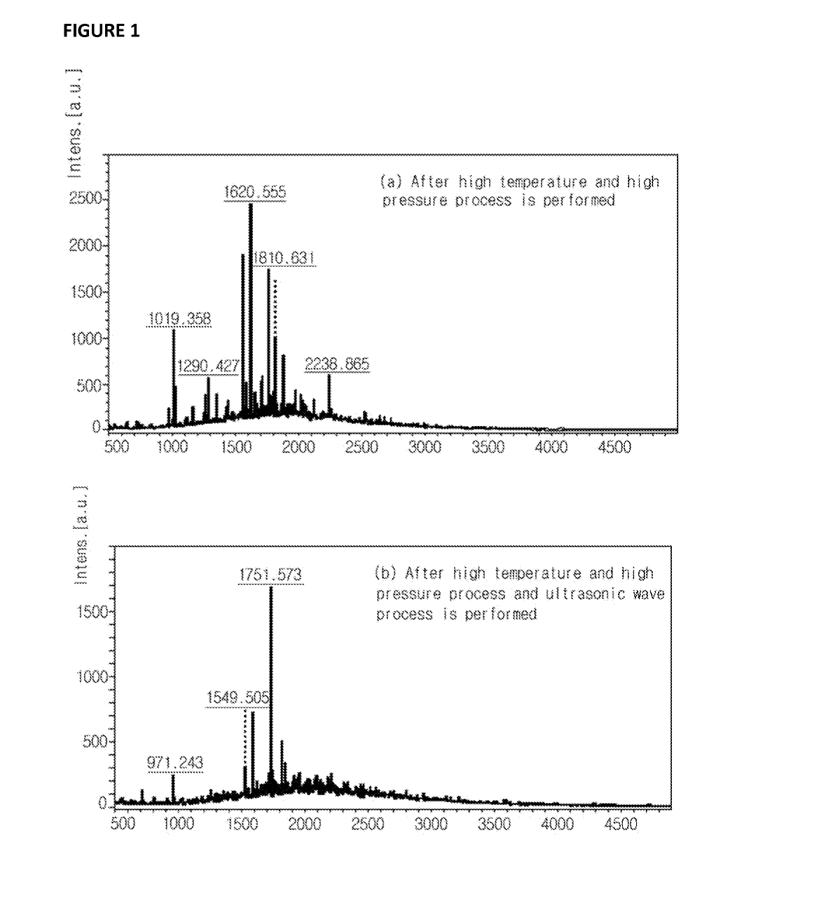

The urechis unicinctus extract obtained by way of the above mentioned procedures features in that the effective components contained in the urechis unicinctus are formed of low molecular weight compounds. According to the experiments of an embodiment of the present invention, the molecular weights of the substances contained in each extract are measured with respect to the occasion that the urechis unicinctus is processed at a high temperature and high pressure and the occasion that the urechis unicinctus is processed at high temperature and high pressure and with ultrasonic waves. As a result, the occasion that the urechis unicinctus is processed with the ultrasonic waves at a high temperature and high pressure seems formed of low molecular weight compounds as compared to the occasions that the urechis unicinctus is processed with only at a high temperature and high pressure. (Refer to FIG. 1).

Therefore, the inventors of the present invention could expect that the pharmaceutical component contained in the urechis unicinctus is changed to the forms of low molecular weight compounds by way of the high temperature and high pressure and the ultrasonic wave process and is administrated or treated, so they can be faster, more easily absorbed, thus inducing the targeted effects.

Meanwhile, the urechis unicinctus extract obtained by the above mentioned method is processed at a temperature of 100˜130° C. and a pressure to of 1 Kgf/cm² for 20˜40 minutes and is further processed with an ultrasonic wave at a frequency 35˜45 KHz for 50˜70 minutes, so the urechis unicinctus processed in the above mentioned method has been proved to have a good sexual function enhancement effect.

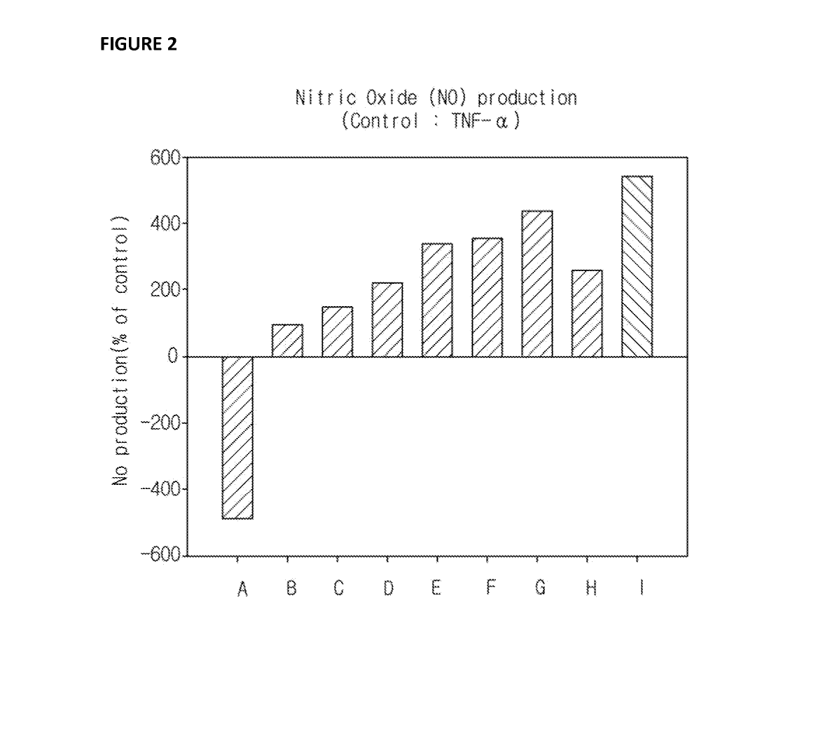

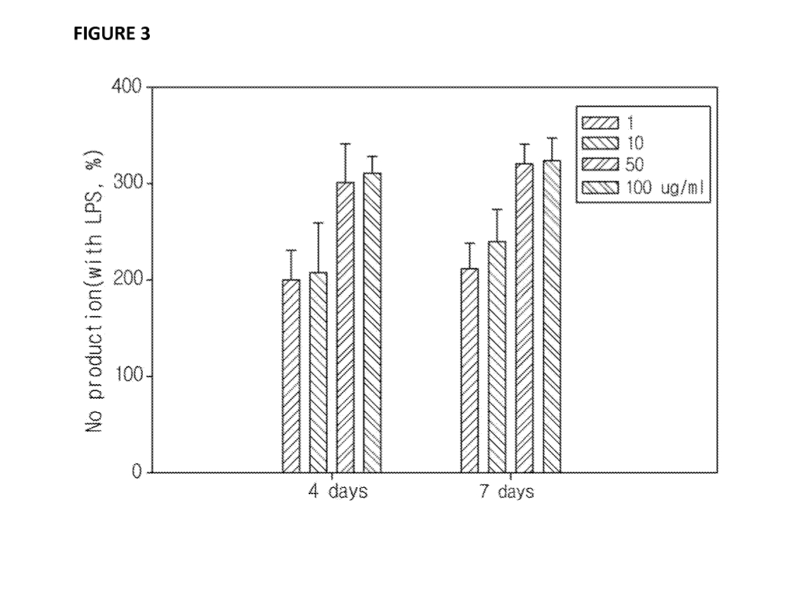

According to an embodiment of the present invention, the generation of the nitrogen monoxide in the cells is measured with respect to the urechis unicinctus extracts obtained by the method of the present invention, As a result, the urechis unicinctus extract obtained by means of the method of the present invention has a function of enhancing the generation of nitrogen monoxide in proportion to the process concentration. (Refer to FIGS. 2 and 3).

The generation of the nitrogen monoxide is measured as a sexual function improvement index because when a male get sexually excited, nitrogen monoxide is generated from a specific neural cell and helps expand the blood vessels connected to a corpus spongiosum of an erection tissue, thus erecting the penis. Viagra which is an erection dysfunction therapy agent is directed to curing an erection dysfunction in such a way to delay the decomposition of nitrogen monoxide.

So, it has been proved that the urechis unicinctus extract of the present invention helps produce nitrogen monoxide, thus effectively enhancing the erection.

In addition, in a preferred embodiment of the present invention, it has been studied if the urechis unicinctus extract of the present invention helps affect the GTP generation in the cells of the smooth muscle.

The sexual stimulation in the body causes a reaction of increasing the input amount of blood while relieving the smooth muscles of the corpus cavernosum. The medium factor of such reaction is nitrogen monoxide. The nitrogen monoxide activates guanylyl cyclase and consequently generates cGMP (Cyclic GMP) from GTP. In addition, the cyclic GMP cause a relief of the smooth muscle, and the erection is promoted thanks to the pressure of vein.

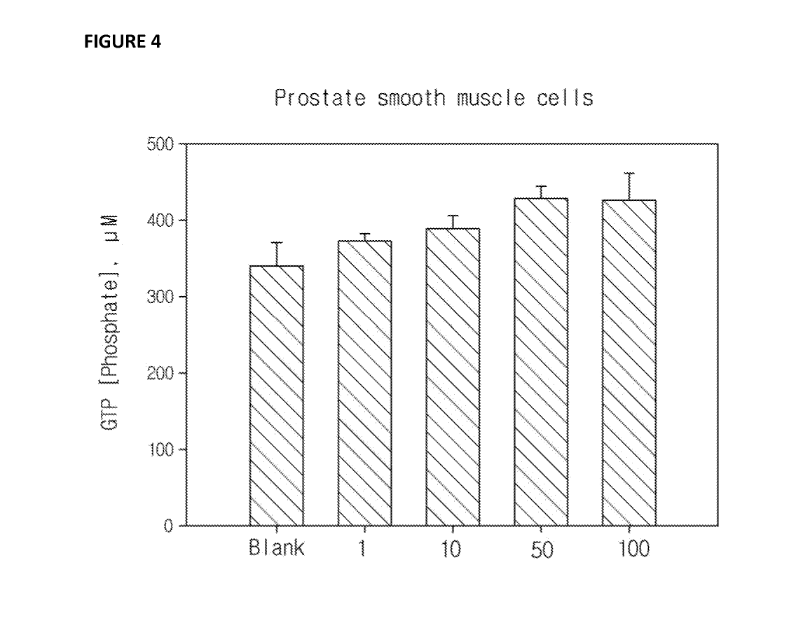

Therefore, a result of the studies if the urechis unicinctus of the present invention can promote the GTP generation from the smooth muscle cells, as shown in FIG. 4, it has been proved that the GTP generation increases in proportion to the process concentration of the extract.

In addition, the urechis unicinctus prepared by the method of the present invention features in that the secretion of the testosterone which is a male hormone increases.

The testosterone which is a male hormone has been proved to adjust the hypothalamus of brain related with the sexual function as well as the erection function of a penis while adjusting the generation of nitric oxide which is a key element helping erect the penis and maintain the erected penis. So, when the body lacks testosterone, an erection dysfunction occurs. In this case, even an erection dysfunction therapy agent is administrated, no effects are obtained.

Therefore, the urechis unicinctus extract of the present invention has an excellent function of effectively enhancing the secretion of testosterone, so it can be used as the sexual function improvement and erection dysfunction therapy agent.

As explained above, the inventors of the present invention have proved that the urechis unicinctus, in details, the urechis unicinctus extract obtained by way of the high temperature and high pressure process and the ultrasonic wave process is quite useful as an effective substance for the sake of the improvements of a sexual function. In other words, as compared to the urechis unicinctus obtained using an ordinary solution which is not processed with a high temperature and high pressure and ultrasonic waves and the urechis unicinctus extract obtained only by way of the high temperature and high pressure process, the urechis unicinctus extract obtained by way of a step in which the urechis unicinctus is processed at a temperature of 100˜130° C. at a pressure of 1 Kgf/cm² for 20˜40 minutes and a step in which it is processed at a frequency of 35˜45 KHz for 50˜70 minutes has showed much better effects in the sexual function improvements.

Therefore, the present invention can make it possible to provide a pharmaceutical composition for improving sexual functions which contains as an effective component the urechis unicinctus extract which is obtained at a temperature of 100˜130° C. at a pressure of 1 Kgf/cm² for 20˜40 minutes for 20˜40 minutes and at a frequency of 35˜45 KHz for 50˜70 minutes.

Meanwhile, the composition of the present invention might contain in a single form an urechis unicinctus extract of the present invention or might further contains a carrier which is approved for the pharmaceutical purpose, an excipient or a diluent. In the above, the pharmaceutically effective amount means the amount enough to improve the sexual function and to cure the erection dysfunction. The urechis unicinctus extract might contain at a concentration of 1 μg/ml˜100 μg/ml with respect to the composition.

In addition, the pharmaceutically effective amount of the urechis unicinctus extract of the present invention is 0.5˜100 mg/day/weight kg, preferably 0.5˜10 mg/day/weight kg; however the above mentioned pharmaceutically effective amount might change depending on the severity of a sexual function disorder, the age of a patient, weight, health state, sex, administration passage and curing period.

In addition, the pharmaceutical approved composition is referred to a composition which does not cause a stomach disorder, an allergic reaction such as dizziness or a similar reaction when the composition is approved for the biological purpose and when it is administrated to a human being. As the examples of the carrier, the excipient and diluent, there are lactose, dextrose, sucrose, sorbitol, mannitol, xylitol, erythritol, maltitol, starch, acacia rubber, alginate, gelatin, calcium phosphate, calcium silicate, cellulose, methyl cellulose, polyvinyl pyrrolidone, water, methylhydroxybenzoate, prophylhydroxybenzoate, talc, magnesium stearate and minerals. In addition, a filler, an anti-cohesive agent, a lubricant, a moisturizer, perfume, emulsifier and antiseptic might be further added.

The composition of the present invention can be formulated in a known way so that the quick, continuous or delayed discharge of the active component can be possible after it is administrated to a mammal. The composition can be formulated in a form of a powder, a granule, a tablet, an emulsion, a syrup, an aerosol, a smooth or solid gelatin capsule, a sterilization injection liquid, a sterilization powder, etc.

The composition according to the present invention might be administrated by way of an oral administration, a skin administration, a subcutaneous administration, a vein or muscle administration, and the dose of the active component might properly change depending on various factors such as an administration way, the age of a patient, sex, weight and the severity of a patient. In addition, the sexual function enhancement composition according to the present invention might be administrated in combination with known compounds which have sexual function improvement effects or erection dysfunction therapy effects.

In addition, the present invention might provide an erection dysfunction therapy agent containing an urechis unicinctus extract, and the erection dysfunction therapy agent is not limited to the disclosed contents, and it can be formulated in a dispersed form, a tablet form, a capsule form, an injection liquid form, a smooth capsule form, a pill or a granule form.

The urechis unicinctus extract according to the present invention can be stably administrated into a human body since it does not have toxicity to cells and does not cause side effects, so the urechis unicinctus extract of the present invention can be used for the purpose of food compositions helping enhance sexual functions.

Therefore, the sexual function improvement food composition containing the urechis unicinctus extract as an effective component can be usefully used as foods such as a principle of a food, a sub-material, a food additive, a functional food or beverage which can help improve or enhance the sexual functions.

The term “food” in the present invention represents natural or processed goods which contain one or more nutrient. Preferably, it means a thing which can be eatable after it is somehow processed. As a conventional meaning, the term represents foods, food additives, functional foods and beverages.

As the foods to which the composition of the present invention can be added, there are various foods, beverages, gum, tea, combined vitamins, functional foods, etc. The foods adapted to the present invention comprise special nutrition foods (for example, processed milk goods, foods for infant and kids), processed meat goods, fish flesh goods, tofu, starch gel, noodles (for example, ramen, noodles, etc), breads, health assistant foods, sauce foods (for example, soy sauce, soybean paste, mixed paste, etc.), sauces, biscuits (for example, snack), candies, chocolates, gums, ice cream, processed milk goods (for example, fermented oil, cheese, etc.), other processed foods, kimchi, salted foods (various kimchis, salted vegetables), beverage (for example, fruit beverage, vegetable beverage, bean oils, fermented beverage, etc.), natural sauces (dried ramen soup), etc. and they are not limited thereto. The above listed foods, beverages or food additives might be manufactured in the conventional way.

In addition, the term “functional food” represents a food group which is value-added to have a desired function and reproduction depending on the function of a corresponding food with the aid of physical, biochemical and bioengineering techniques or a food which is designed or processed to add to biological body a biological adjusting function on a biological protection rhythm adjustment, a disease prevention and recovery that a food composition has. The functional food represents in details a health functional food. The functional food might include a food sub-additive which is approved for the purpose of food engineering. The functional food further includes a proper carrier, excipient and diluent which are conventionally used for the sake of a functional food manufacture.

In addition, the term “beverage” represents a thing that people drinks for the purpose of killing thirst or enjoying the taste and includes a functional beverage. The beverage is not limited to other components except for the composition of the present invention contained as necessary components at set ratios, and the beverage might contain various flavoring agents or natural carbohydrate like known beverages as added components.

The food containing a composition of the present invention, except for the above listed foods, comprises various nutrients, vitamins, mineral (electrolysis), flavoring agents such as combined flavoring agents and natural flavoring agents, a coloring agent and a filler (cheese, chocolate, etc.), pectic acid and its salt, organic acid, protective colloid thickener, pH adjuster, stabilizer, antiseptic, glycerin, alcohol, carbonated soft drink, etc. The above mentioned component can be added in a single form or can be added in combination.

In the food which contains the composition of the present invention, the composition according to the present invention is contained 0.001 weight % to 90 weight % with respect to the whole weight of the food, preferably, is contained 0.1 weight % to 40 weight %, and in case of beverage, it is contained 0.001 g to 2 g with respect to 100 ml, preferably it is contained at a ratio of 0.01 g to 0.1 g. When it is taken in for long time for the purpose of health and sanitary or for the purpose of health control, the intake range might be below the above mentioned range. Since the effective components do not have any problems in terms of safety, it can be taken more than the above mentioned range, so the intake range is not limited.

The present invention provides a method for curing an erection dysfunction of a patient which comprises a step for administrating an urechis unicinctus extract of a pharmaceutically effective amount according to the present invention to a patient who suffers from an erection dysfunction.

The present invention provides a method for enhancing an erection of an object which comprises a step for administrating an urechis unicinctus extract of a pharmaceutically effective amount to an object which has an erection power lowered.

In addition, the applicable range of the method of the present invention includes a vertebrata, preferably, a mammal. More preferably, it includes an experimental animal such as a mouse, a rabbit, guinea pig, a hamster, a dog, a cat, etc, and most preferably it includes an anthropoid animal such as chimpanzee, gorilla, etc. The method of administrating the extract of the present invention comprises an oral or parenteral administration like the earlier explained administration ways, and in case of the parenteral administration, it might be performed by way of an intraperitoneal injection, a rectum injection, a subcutaneous injection, a vein injection or a muscle injection

The preferred embodiments of the present invention will be described in details. The following embodiment is provided for the detailed descriptions only, and does not limit the scope of the present invention.

### Embodiment 1

Preparation of Urechis Unicinctus Extract in Ultrasonic Wave Process

10 Kg of urechis unicinctus was obtained from a sea market of Gwangari, Suyoung-gu, Busan city, Korea and was cut with scissors into pieces of 0.8˜1 cm, and the cut pieces of urechis unicinctus were processed at a temperature of 121° C. at a pressure of 1 Kg/cm² for 30 minutes using Autoclave hs1212 (Hanshin medical), and were processed with ultrasonic waves at a frequency of 40 KHz for 1 hour, thus preparing an urechis unicinctus extract. FIG. 1 shows the distributions of the molecular weights of a component contained in the urechis unicinctus after it was processed only at a high temperature and a high pressure and it was processed at a high temperature and a high pressure and then was processed at an ultrasonic wave. As a result, it was proved that the sample processed at a high temperature and a high pressure and with ultrasonic waves was changed to a low molecular weight compound.

### Embodiment 2

Nitrogen Monoxide Generation Analysis

The inventors of the present invention checked the generations of nitrogen monoxide so as to prove whether the urechis unicinctus extract obtained by way of a high temperature and a high pressure process and a ultrasonic wave process had a sexual function improvement effect or not. Thanks to the generation of nitrogen monoxide, GTP is generally changed to cGMP, so the artery and corpus cavernosum expanded, thus raising the internal pressure, which contributes to erection. So, the inventors of the present invention processed the urechis unicinctus extract of the present invention by the concentration and time with respect to the PC-2 cells which are the prostate cell line and measured the changes in generations of nitrogen monoxide.

In more details, the PC-3 prostate cancer cells) cells were divided into 96-well plates and were cultured using the DMEN culture medium which did not have phenol red. The urechis unicinctus extract of the present invention was processed at a concentration of 100 μg/ml for 1 hour before nitrogen monoxide was induced, and the LPS of 1 mg/ml was processed with respect to the above mentioned cells and was cultured for 48 hours and the nitrogen monoxide was induced. After the cultivation, the cell culture liquid of 50 ml was mixed with a Griess reagent with the same volume and was reacted for 15 minutes, and the absorbance was measured using ELISA micro plate reader (Tecan Austria GmbH, Austria) at 550 nm. In addition, the inventors of the present invention purchased the heads of sea mustard, sea squirt, comb pen shell, turbon shell, squid, sea tangle, scallop and oyster and obtained the extracts from them in the manner in the embodiment 1, and the generations of nitrogen monoxide were checked in the same manner as earlier.

As a result, as shown in FIG. 2, the marine products except for the heads of sea mustard, there were the generations of the nitrogen monoxide, among which it was proved that the urechis unicinctus showed the highest generations of the nitrogen monoxide as compared to the other marine products.

In addition, the urechis unicinctus extracts according to the present invention were processed on the cell lines at the concentrations (1, 10, 50, 100 ug/ml) for 4 days and 7 days, and the generations of the nitrogen monoxide were checked. As shown in FIG. 3, the generations of the nitrogen monoxide rose depending on the process concentration and process time.

The inventors of the present invention checked the generations of the nitrogen monoxide in the same manner as earlier with respect to the urechis unicinctus extract obtained by way of a high temperature and high pressure process and an ultrasonic wave process in the embodiment 1, the urechis unicinctus extract obtained by way of the high temperature and high pressure process only and the urechis unicinctus which was not processed.

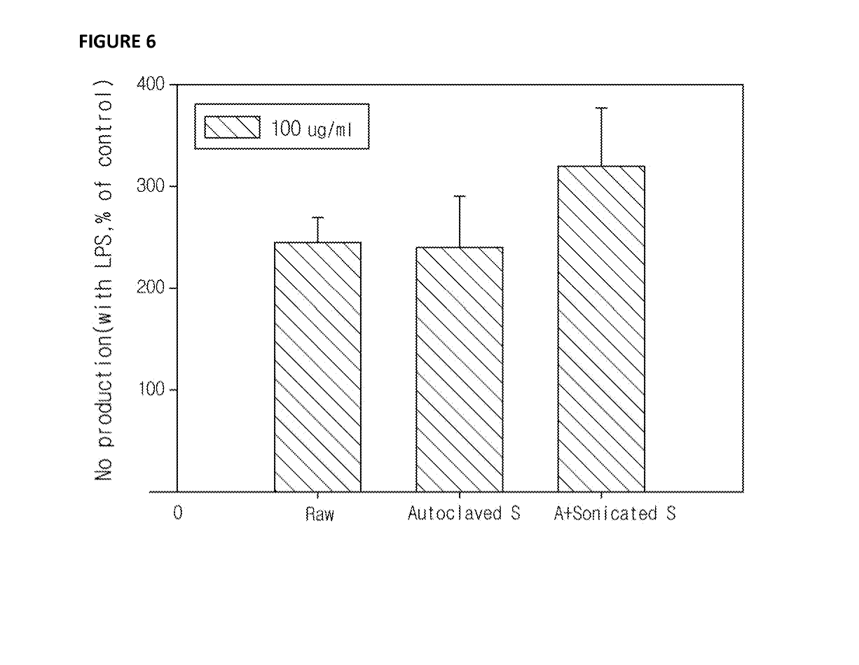

As a result, as shown in FIG. 6, in case of the group which was processed by way of a high temperature and high pressure process, the generations of the nitrogen monoxide was similar with the group of the urechis unicinctus which were not processed; however the group which was processed at a high temperature and a high pressure and then with ultrasonic waves showed the significantly raised generations of the nitrogen monoxide as compared to the other experimental groups.

The above results were obtained since the urechis unicinctus was changed to low molecular weight compounds for the sake of easier absorbance into a human body by way of the high temperature and high pressure process and the ultrasonic wave process. The sample before the process was performed, namely, the urechis unicinctus which was not processed and the urechis unicinctus which was processed at a high temperature and a high pressure are formed of high molecular weight compound in the protein contained in the urechis unicinctus, so they cannot be well absorbed into the human body. Therefore, the high temperature and high pressure process and the ultrasonic wave process in the present invention have been proved to help change the useful proteins contained in the urechis unicinctus to low molecular weight compounds, so it is possible to obtain an urechis unicinctus which has good pharmaceutical effects.

### Embodiment 3

Analysis on how the Urechis Unicinctus Extract of the Present Invention Affects the Generation of GTP in the Prostate Smooth Muscle Cell

The GTPase analysis was conducted so as to check how the urechis unicinctus extract prepared by the method of the present invention affects the generation of GTP in the prostate smooth muscle cells. In other words, the urechis unicinctus extract of the present invention was processed on the prostate smooth muscle cells by the concentrations (1, 10, 50, 100 ug/ml) and was cultivated for 24 hours, and the GTPase activity in the cell was measured using QuantiChrom™ ATPase/GTPase of BioAssay System company.

As a result, as shown in FIG. 4, it was proved that the concentration of the GTP was raised in proportion to the process concentration of the urechis unicinctus extract. So, seeing the above mentioned method, the inventors of the present invention was convinced that the urechis unicinctus extract obtained by the method of the present invention had increased the amount of GTP in the prostate smooth muscles, and the raised GTP had a high chance to convert to the cGMP.

### Embodiment 4

Analysis on how the Urechis Unicinctus Extract of the Present Invention Affects the Secretion of Male Hormone.

The inventors of the present invention processed the urechis unicinctus extract of the present invention on the prostate smooth muscle cells with the concentrations (1, 10, 50, 100 ug/ml) and cultivated them for 24 hours so as to check whether or not the urechis unicinctus extract of the present invention affected the secretions of the testosterone which is a male hormone and then measured the amount of the secreting testosterone using the testosterone EIA kit (Cayman Chemical Company, Ann Arbor, Mich.). At this time, the concentration of the testosterone was calculated along the standard concentration curve using the testosterone standard solution provided in the EIA kit.

As a result, it was proved that the urechis unicinctus extract of the present invention helped increase the secretion of the testosterone, which is a male hormone, in proportion to the process concentration. It is told that the testosterone, which is a male hormone, is related to the sexual desire and erection of a penis; however a male ages, the secretion of the testosterone hormone of a male decreases, and the sexual desired reduces, and the erection dysfunction occurs.

However, in the present invention, it has been proved that the urechis unicinctus extract obtained by way of the high temperature and high pressure process and then the ultrasonic wave process has a strong activation helping promote the secretion of male hormones, so it can be used for the purpose of improving the sexual functions.

### Embodiment 5

Analysis on how the Urechis Unicinctus Extract of the Present Invention Affects the Secretion of a Male Hormone

In order to check the instant toxicity with respect to the urechis unicinctus extract obtained according to the present invention, the following experiments were conducted. There were prepared three groups each consisting of 4 female and male mouse of 4 weeks ages of the SPF (Specific Pathogens Free), and they were grown in an animal chamber under the condition of a temperature of 22±3° C., a moisture of 55±10% and a lighting of 12 L/12 D. The mouse was smoothened for 1 week before they were used for the experiments. The feeds for the experimental animals (for moue and rat, Dyets, USA) and drinks were sterilized and supplied freely.

The urechis unicinctus extract obtained in the embodiment 1 of the present invention was prepared at a concentration of 50 mg/ml with respect to 0.5% tween and was orally medicated to 0.02 ml (100 mg/kg) per 20 g of the mouse weight, 0.2 ml (500 mg/kg) and 0.5 ml (1,000 mg/kg), and the side effects or deaths were observed for 7 days after the medication. In other words, the changes of the ordinary symptom and the deaths were observed one time every in the morning and the afternoon until 7th day from the next day of the medication. In addition, the animals were killed at the 7th day and the internal organs were checked visually after anatomy. The changes of the weight were checked every another day from the date of medication, so the weight reductions of the animals due to the urechis unicinctus extract of the present invention were observed.

As a result, all the mouse to which the urechis unicinctus extract was medicated did not show specific symptoms, and no muse were died, and toxicity changes were not found in the weight change, the blood check, the blood biochemical test, the autopsy, etc. As a result, it has been proved that the urechis unicinctus extract of the present invention does not cause any toxicity in the body, so it is a stable substance.

### Embodiment 6

Analysis on the Improvements in the Sexual Function from the Urechis Unicinctus Extract Based on a High Temperature and High Pressure Process is Conditions

The inventors of the present invention conducted the following experiments so as to check the optimization in the manufacture of the urechis unicinctus extract of the present invention. In other words, as shown in Table 1, the urechis unicinctus extract obtained by way of the high temperature and high pressure process and the ultrasonic wave process was medicated to the mouse, and the erection duration strength was measured.

In more details, the male rat of Sprague Dowley (Spraque-Dowley Company, Samtacho Bio Korea) of 250˜35 g was grown at a temperature 22±2° C. and a relative moisture of 50±10% with a lighting cycle (07:00 turn on˜19:00 turn off) of a fluorescent light, and each urechis unicinctus extract of Table 1 was medicated to the mouse (dose was 20 mg/kg of the urechis unicinctus extract), and the erection duration strength was measured after 40 minutes. The erection duration strength was measured by way of a reaction evaluation of the penis erection with respect to a stimulated nerve of corpus cavernosum. The internal pressure of the corpus cavernosum was measured based on the stimulated nerve of the corpus cavernosum (stimulus: 10 Hz, 3V, ms, 1 min), and the ratios of the maximum internal pressure of the corpus cavernosum (ICP: Intracranial Pressure) and the blood pressure (MAP: Mean Alterialpressure) were recorded for 5 minutes, and the maximum values of them were compared for each group.

TABLE 1
Guaranteed condition of urechis unicinctus extract

| | High temperature | Ultrasonic | |
|---|---|---|---|
| | and high pressure | wave | Maximum penis |
| | condition | condition | erection power |
| Conditions of | Temperature of 121°, | 30 KHz | 84.46 ± 4.9% |
| the present | pressure of 1 Kgf/cm² | | |
| invention | | | |
| Comparison | Temperature of 90°, | 40 KHz | 54.20 ± 3.2% |
| example 1 | pressure of 0.8 Kgf/cm² | | |
| Comparison | Temperature of 150°, | 50 KHz | 60.58 ± 2.2% |
| example 2 | pressure of 2 Kgf/cm² | | |

As a result, as shown in Table 1, it was proved that the urechis unicinctus extract obtained based on the high temperature and high pressure and ultrasonic wave process conditions of the present invention has the most excellent sexual function activations.

So, the inventors of the present invention has recognized that the process adapted to obtain the urechis unicinctus extract of the present invention has the best conditions for the purpose of obtaining urechis unicinctus extract having the most effective sexual function activations.

### Preparation Example 1

<1-1> Preparation of Powders

| | |
|---|---|
| urechis unicinctus extract of the present invention | 2 g |
| lactose | 1 g |

The above components are mixed, and the powders are prepared by filling it into a sealed pocket.

<1-2> Preparation of Tablets

| | |
|---|---|
| urechis unicinctus extract of the present invention | 100 mg |
| Corn starch | 100 mg |
| lactose | 100 mg |
| stearic acid magnesium | 2 mg |

The above components are mixed, and the mixed components are tabled-formed based on the conventional tablet preparation method, thus preparing the tablets.

<1-3> Preparation of Capsules

| | |
|---|---|
| urechis unicinctus extract of the present invention | 100 mg |
| Corn starch | 100 mg |
| lactose | 100 mg |
| stearic acid magnesium | 2 mg |

The above components are mixed, and the mixed components are filled into a gelatin capsule based on the conventional capsule preparation method, thus preparing the capsules.

<1-4> Preparation of Pills

| | |
|---|---|
| urechis unicinctus extract of the present invention | 1 g |
| lactose | 1.5 g |
| glycerin | 1 g |
| xylitol | 0.5 g |

The above components are mixed, and one pin weighs 4 g.

### Preparation Example 2

### Preparation of Foods

The health foods containing the urechis unicinctus of the present invention as an effective component is prepared as follows.

<2-1> Preparations of Drinks

| | | |
|---|---|---|
| honey | 522 | mg |
| thiocticacid | 5 | mg |
| nicotinamide | 10 | mg |
| sodium hydrochloric acid riboflavin | 3 | mg |
| hydrochloric acid pyridoxine | 2 | mg |
| inositol | 30 | mg |
| orotic acid | 50 | mg |
| urechis unicinctus extract of the present invention | 1.28 | mg |
| water | 200 | ml |

The beverage was prepared with the above listed composition and contents in the ordinary method.

<2-2> Preparation of Candies

| | | |
|---|---|---|
| sugar | 50~60 | weight % |
| water starch syrup | 39.26~4.66 | weight % |
| urechis unicinctus extract of present invention | 0.24~0.64 | weight % |
| orange flavor | 0.1 | weight % |

The candies were prepared with the above listed compositions and contents in the ordinary method.

As the present invention may be embodied in several forms without departing from the spirit or essential characteristics thereof, it should also be understood that the above-described examples are not limited by any of the details of the foregoing description, unless otherwise specified, but rather should be construed broadly within its spirit and scope as defined in the appended claims, and therefore all changes and modifications that fall within the meets and bounds of the claims, or equivalences of such meets and bounds are therefore intended to be embraced by the appended claims.

1. A method for treating an erectile dysfunction in a mammal in need thereof which comprises administering to said mammal an effective amount of an urechis unicinctus extract and yielded by way of a process at a temperature 100˜130° C. at a pressure of 1 Kgf/cm² for 20˜40 minutes and then with an ultrasonic wave at a frequency 35˜45 KHz for 50˜70 minutes.

2. A pharmaceutical composition for a sexual function improvement comprising; an urechis unicinctus extract contained as an effective component and yielded by way of a process at a temperature 100˜130° C. at a pressure of 1 Kgf/cm² for 20˜40 minutes and then with an ultrasonic wave at a frequency 35˜45 KHz for 50˜70 minutes.

3. The composition of claim 2, wherein the urechis unicinctus extract is contained the composition at a concentration of 1˜100 ug/ml.

4. The composition of claim 2, wherein the composition is provided in a form of powders, tablets, capsules, injection liquid, smooth capsules, pills or granule.

5. A health food for a sexual function improvement, comprising:
an urechis unicinctus extract contained as an effective component and yielded by way of a process at a temperature 100˜130° C. at a pressure of 1 Kgf/cm² for 20˜40 minutes and then with an ultrasonic wave at a frequency 35˜45 KHz for 50˜70 minutes.

6. The food of claim 5, wherein the urechis unicinctus is contained at a concentration of 1˜100 ug/ml.

7. A method for preparing an urechis unicinctus having a sexual function improvement activation, comprising:
a step for cutting urechis unicinctus into pieces each of 0.8 cm˜1.0 cm;
a step for processing the cut urechis unicinctus at a temperature of 100˜130° C. at a pressure of 1 Kgf/cm² for 20˜40 minutes; and
a step for performing an ultrasonic wave process at a frequency of 35˜45 KHz for 50˜70 minutes.
